(19) United States
(12) Patent Application Publication
(10) Pub. No.: US 2011/0177157 A1
(43) Pub. Date: Jul. 21, 2011

(54) Compositions for Controlled Delivery of Pharmaceutically Active Compounds

(75) Inventor: Murthy V.S.N. Yerramilli, Falmouth, ME (US)

(73) Assignee: IDEXX Laboratories, Inc., Westbrook, ME (US)

(21) Appl. No.: 12/977,682

(22) Filed: Dec. 23, 2010

Related U.S. Application Data
(60) Provisional application No. 61/296,134, filed on Jan. 19, 2010.

(51) Int. Cl. A61K 9/127; A61K 47/16; A61K 47/22; A61K 47/18

(52) U.S. Cl. 424/450; 514/784

(57) ABSTRACT

Pharmaceutical compositions comprising (i) a dibasic amino acid ester or dibasic amino acid amide; (ii) a dicarboxylic acid; (iii) a pharmaceutically active compound or pharmaceutically acceptable salt thereof; and (iv) a pharmaceutically acceptable organic solvent. The pharmaceutical compositions form a depot when administered to an animal by injection that releases the pharmaceutically active compound over time.

### 1. CROSS REFERENCE TO RELATED APPLICATIONS

This application claims the benefit of U.S. Provisional Application Ser. No. 61/296,134, filed on Jan. 19, 2010, the contents of which are expressly incorporated herein.

### 2. STATEMENT REGARDING FEDERALLY SPONSORED RESEARCH OR DEVELOPMENT

Not Applicable.

### 3. INCORPORATION BY REFERENCE OF MATERIAL SUBMITTED ON A COMPACT DISC

Not Applicable.

### 4. FIELD OF THE INVENTION

The invention relates to sustained-release pharmaceutical compositions and to methods of administering pharmaceutically active compounds to an animal using the sustained-release pharmaceutical compositions.

It is often desirable to administer drugs using controlled- or sustained-release formulations that can maintain therapeutic blood levels of the drug over extended periods of time. These controlled release formulations reduce the frequency of dosing, for enhanced convenience and compliance, and also reduce the severity and frequency of side effects. By maintaining substantially constant blood levels and avoiding blood level fluctuations of the drug, such as are associated with conventional immediate release formulations that are administered several times a day, controlled- or sustained-release formulations can provide a better therapeutic profile than is obtainable with conventional immediate release formulations.

Known methods for controlled- or sustained-drug release include implanted devices, such as osmotic pumps and drug dispersed in a biocompatible polymer matrix, which can be implanted, administered orally, or injected. Examples of biocompatible polymers used in such applications include poly(lactic acid) and poly(lactic acid-co-glycolic acid). The polymer typically undergoes slow hydrolysis in vivo to continually release the entrapped drug over time. The polymer degradation products are non-toxic and absorbed or metabolized by the body. For example, when the biocompatible polymer is poly(lactic acid) or poly(lactic acid-co-glycolic acid), the degradation products are the parent acids, lactic acid and glycolic acid, which are absorbed by the body.

There remains a need in the art, however, for drug containing pharmaceutical compositions, suitable for injection, wherein the formulation provides controlled- or sustained-release of the drug.

### 5. SUMMARY OF THE INVENTION

The invention relates to pharmaceutical compositions and methods of treating or preventing a condition in an animal by administering the pharmaceutical composition to the animal. Typically, the administration is by injection. The pharmaceutical compositions comprise (i) a dibasic amino acid ester or dibasic amino acid amide; (ii) a dicarboxylic acid; (iii) a pharmaceutically active compound or pharmaceutically acceptable salt thereof; and (iv) a pharmaceutically acceptable organic solvent.

In one embodiment, the pharmaceutical composition forms a precipitate when injected into water. The invention is further directed to the solid precipitate. The precipitate can be isolated and formulated into a solid dosage form. The solid dosage form can be administered orally.

In one embodiment, the pharmaceutical composition forms a depot when administered to an animal by injection.

The invention further relates to a pharmaceutical composition comprising micelles or liposomes suspended in an aqueous solvent, wherein the micelles or liposomes comprise (i) a dibasic amino acid ester or dibasic amino acid amide; (ii) a dicarboxylic acid; and (iii) a pharmaceutically active compound or pharmaceutically acceptable salt thereof.

### 6. BRIEF DESCRIPTION OF THE DRAWINGS

FIG. 1 illustrates a possible structure for the precipitate that is formed when the pharmaceutical composition of the invention is injected into water. The illustration depicts a pharmaceutically active compound (indicated by the solid sphere) entrapped within an extended salt structure that is formed between a dibasic amino acid ester and a N-acylated amino acid that is a dicarboxylic acid.

### 7. DETAILED DESCRIPTION OF THE INVENTION

The invention relates to pharmaceutical compositions for administering pharmaceutically active compounds. The pharmaceutical compositions provide sustained- or controlled-release of the pharmaceutically active compound. The invention further relates to methods of treating or preventing a condition in an animal comprising administering to an animal in need thereof a pharmaceutical composition of the invention.

The pharmaceutical compositions comprise:

(i) a dibasic amino acid ester or dibasic amino acid amide;

(ii) a dicarboxylic acid;

(iii) a pharmaceutically active compound or pharmaceutically acceptable salt thereof; and

(iv) a pharmaceutically acceptable organic solvent.

In one embodiment, the dibasic amino acid ester is a dibasic amino acid vitamin ester.

In one embodiment, the dicarboxylic acid is an N-acylated amino acid, wherein the side chain of the amino acid contains an acidic functional group.

The invention further relates to a pharmaceutical composition comprising: (i) a dibasic amino acid ester or dibasic amino acid amide; (ii) a dicarboxylic acid; and (iii) a pharmaceutically active compound or pharmaceutically acceptable salt thereof. In one embodiment the pharmaceutical composition is a solid.

The invention further relates to a pharmaceutical composition comprising micelles or liposomes suspended in an aqueous solvent, wherein the micelles or liposomes comprise

(i) a dibasic amino acid ester or dibasic amino acid amide;

(ii) a dicarboxylic acid; and

(iii) a pharmaceutically active compound or pharmaceutically acceptable salt thereof.

### Definitions

The term “pharmaceutically acceptable organic solvent,” as used herein, means an organic solvent that when administered to an animal does not have undue adverse effects such as excessive toxicity, irritation, or allergic response commensurate with a reasonable benefit/risk ratio. Preferably, the pharmaceutically acceptable organic solvent is a solvent that is generally recognized as safe (“GRAS”) by the United States Food and Drug Administration (“FDA”).

The term “water miscible organic solvent,” as used herein, means an organic solvent that is capable of mixing with water in any ratio without separating into two phases.

The term “water soluble organic solvent,” as used herein, means an organic solvent that has a significant level of solubility in water. Typically, a water soluble organic solvent is soluble in water in an amount of at least about 5 percent by weight, preferably at least about 10 percent by weight, more preferably at least about 20 percent by weight, and most preferably at least about 50 percent by weight. For example, triacetin is considered a water soluble solvent since it is soluble in water at a ratio of about 1:14.

The phrase “drug depot,” as used herein, means a precipitate that includes the pharmaceutically active compound formed within the body of a treated animal that releases the pharmaceutically active compound over time.

The phrase “forms a precipitate,” as used herein, means that the pharmaceutical composition forms a precipitate, or solid, when injected into water or into a physiological (in vivo) environment. A precipitate is an insoluble solid formed in solution at room temperature in vitro or in a physiological (in vivo) environment. The precipitate can take many forms such as, for example, a solid, a crystal, a gummy mass, or a gel. Preferably, the precipitate is a gummy mass or a gel. A composition of the invention forms a precipitate in water when at least 10% of the composition is retained on a 0.22 μm filter when the composition is injected into water and filtered at 98° F. Typically, to form the precipitate about 1 mL of the pharmaceutical composition is injected into about 5 mL of water.

The phrase “injectable” or “injectable composition,” as used herein, means a composition that can be drawn into a syringe and injected subcutaneously, intraperitoneally, or intramuscularly into an animal without causing adverse effects due to the presence of solid material in the composition. Solid materials include, but are not limited to, crystals, gummy masses, and gels. Typically, a formulation or composition is considered to be injectable when no more than 10%, preferably no more than 5%, more preferably no more than 2%, and most preferably no more than 1% of the formulation is retained on a 0.22 gm filter when the formulation is filtered through the filter at 98° F.

The term “pharmaceutically active compound,” as used herein, means a compound that causes a pharmacological effect in an animal. Typically, the pharmacological effect is treating or preventing a condition in an animal. Unless otherwise specified, the phrase “pharmaceutically active compound” encompasses salts of the pharmaceutically active compound.

The term “condition,” as used herein means an interruption, cessation, or disorder of a bodily function, system, or organ. Representative conditions include, but are not limited to, infections such as bacterial, viral, fungal and, parasitic infections; diseases such as cancer; inflammation; diabetes; and organ failure.

The term “effective amount,” as used herein, means an amount sufficient to treat or prevent a condition in an animal.

The phrase “treating,” “treatment of,” and the like includes the amelioration or cessation of a specified condition.

The phrase “preventing,” “prevention of,” and the like include the avoidance of the onset of a condition or a reduction in the likelihood of the onset of a condition.

The term “animal,” as used herein, includes, but is not limited to, humans, canines, felines, equines, bovines, ovines, porcines, amphibians, reptiles, and avians. Representative animals include, but are not limited to a cow, a horse, a sheep, a pig, an ungulate, a chimpanzee, a monkey, a baboon, a chicken, a turkey, a mouse, a rabbit, a rat, a guinea pig, a dog, a cat, and a human. In one embodiment, the animal is a mammal. In one embodiment, the animal is a human. In one embodiment, the animal is a canine, a feline, an equine, a bovine, an ovine, or a porcine.

The phrase “pharmaceutically acceptable salt,” as used herein, is a salt formed from an acid and a basic group of a pharmaceutically active compounds. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, ben.zenesulfonate,p-toluenesulfonate, and pamoate (i.e., 1,1′-methylene-bis-(2-hydroxy-3-naphthoate)) salts. The term “pharmaceutically acceptable salt” also refers to a salt prepared from a pharmaceutically active compound having an acidic functional group, such as a carboxylic acid functional group, and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)amine, 2-hydroxy-tert-butylamine, or tris-(hydroxymethyl)methylamine, N,N,-di-lower alkyl-N-(hydroxy lower alkyl)-amines, such as N,N,-dimethyl-N-(2-hydroxyethyl)amine, or tri-(2-hydroxyethyl)amine; N-methyl-D-glucamine; and amino acids such as arginine, lysine, and the like.

The term “dibasic amino acid,” as used herein means an amino acid of formula

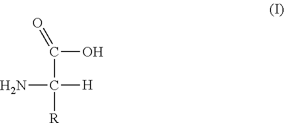

wherein the amino acid side chain, R, includes a basic amine group.

The phrase “C₁-C₂₂ hydrocarbon group,” as used herein means, a straight or branched, saturated or unsaturated, cyclic or non-cyclic, aromatic or non-aromatic, carbocyclic or heterocyclic group having from 1 to 22 carbon atoms. Similar phrases such as, “C₁-C₁₈ hydrocarbon group,” “C₁-C₁₀ hydrocarbon group,” “C₆-C₁₈ hydrocarbon group,” “C₈-C₁₈ hydrocarbon group,” and a “C₁₀-C₁₈ hydrocarbon group” have a similar meaning and mean a straight or branched, saturated or unsaturated, cyclic or non-cyclic, aromatic or non-aromatic, carbocyclic or heterocyclic group having from 1 to 18 carbon atoms, from 1 to 10 carbon atoms, from 6 to 18 carbon atoms, from 8 to 18 carbon atoms, and from 10 to 18 carbon atoms, respectively. Accordingly, the phrase “an acyl group of formula —C(O)—R₅, wherein R₅ is a C₁ to C₂₁ group means an acyl group of formula —C(O)—R₅, wherein R₅ is a straight or branched, saturated or unsaturated, cyclic or non-cyclic, aromatic or non-aromatic, carbocyclic or heterocyclic hydrocarbon group having from 1 to 21 carbon atoms. Representative acyl groups of formula —C(O)—R₅, wherein R₅ is an unsubstituted C₁ to C₂₁ group include, but are not limited to, acetyl, propionyl, butanoyl, hexanoyl, caproyl, laurolyl, myristoyl, palmitoyl, stearoyl, palmioleoyl, oleoyl, linoleoyl, linolenoyl, and benzoyl.

The term “vitamin,” as used herein, is its art recognized meaning, i.e., nutrients required in tiny amounts for essential metabolic reactions in the body. The term vitamin, however, does not include other essential nutrients such as dietary minerals, essential fatty acids, or essential amino acids, nor does it encompass the large number of other nutrients that promote health but that are not essential for life.

The phrase “residue of a vitamin,” as used herein, means a vitamin that has a hydroxyl (i.e., —OH group) wherein the hydrogen of the hydroxyl group is removed. For example, if the formula of the vitamin is H—O—R₁, the formula for the “residue of the vitamin” will be —OR₁. The phrase “residue of an acidic vitamin,” as used herein, means a vitamin that has a carboxylic acid group (i.e., —C(O)OH group) wherein the —OH group of the carboxylic acid functionality is removed. For example, if the formula of the vitamin is R₁—C(O)OH, the formula for the “residue of an acidic vitamin” will be R₁—C(O)—.

The phrase “acidic pharmaceutically active compound,” as used herein, means a pharmaceutically active compound that has an acidic functional group, i.e. , a group that is capable of donating a proton to a basic functional group such as an amine group. Representative acidic functional group include, but are not limited to —COOH (i.e., carboxylic acid groups), —S(O)₂—OH (i.e., sulfonic acid groups), —OP(O)(OR)(OH), —O(P)(OH)₂, —P(O)(OR)(OH), —(P)(OH)₂), —OP(O)(R)(OH), and —P(O)(R)(OH), wherein R is a hydrocarbon group that can optionally be substituted.

The phrase “basic pharmaceutically active compound,” as used herein, means a pharmaceutically active compound that has a basic functional group, i.e., a group that is capable of being protonated. Typically, the basic functional group is an amine group.

The phrase “neutral pharmaceutically active compound,” as used herein means a pharmaceutically active compound that has no net charge. Neutral pharmaceutically active compounds include zwitterions.

The term “hydrophobic pharmaceutically active compound,” as used herein, means a compound that has an octanol-water partition value and/or a octanol water-distribution value of at least about 1, preferably at least about 1.5, more preferably at least about 2, and most preferably at least about 2.5.

The term “hydrophilic pharmaceutically active compound,” as used herein, means a compound that has an octanol-water partition value and/or a octanol water-distribution value of at less than about 1, preferably less than about 0.5, more preferably less than about 0.25, and most preferably less than about 0.1.

The phrase “octanol-water partition value,” or octanol-water partition coefficient,” as used herein, has its art recognized meaning, i.e., the ratio of concentrations of a compound in the two phases of a mixture of water and octanol at equilibrium (Leo A, Hansch C. and Elkins D. (1971), “Partition coefficients and their uses,” Chem. Rev. 71 (6): 525-616; Sangster, James (1997), “Octanol-Water Partition Coefficients: Fundamentals and Physical Chemistry,” Vol. 2 of Wiley Series in Solution Chemistry, Chichester: John Wiley & Sons Ltd.; Moriguchi I., Hirono S., Liu Q., Nakagome I., Matsushita Y. (1992). “Simple method of calculating octanol/water partition coefficient,” Chem. Pharm. Bull. 40 (1): 127-130; Liao Q., Yao J., Yuan S. (2006). “SVM approach for predicting Log P,” Mol. Divers., 10 (3): 301-9; Collander R. (1951). “The partition of organic compounds between higher alcohols and water,” Acta. Chem. Scand., 5: 774-780; and Wasik S. P., Tewari Y. B., Miller M. M., Martire D. E., (1981). “Octanol-Water Partition Coefficients and Aqueous Solubilities of Organic Compounds,” NBS Techn. Rep. 81 (2406): S1-56)). The partition coefficient can be determined by means well known to those skilled in the art. Typically, to measure the partition coefficient of ionizable solutes, the pH of the aqueous phase is adjusted such that the predominant form of the compound is unionized. The logarithm of the ratio of the concentrations of the unionized solute in the solvents is called log P, wherein log P_{oct/water}=log([solute]_{octanol}/[solute^{unionized}]water). A related phrase, “octanol water-distribution value” or “octanol-water distribution coefficient,” as used herein, is the ratio of the sum of the concentrations of all forms of the compound (ionized plus unionized) in each of the two phases. For measurements of distribution coefficient, the pH of the aqueous phase is buffered to a specific value such that the pH is not significantly perturbed by the introduction of the compound. The logarithm of the ratio of the sum of concentrations of the solute's various forms in one solvent, to the sum of the concentrations of its forms in the other solvent is called log D, wherein log D_{oct/water}=log {[solute]_{octanol}/([solute^{ionized}]_{water}+[solute^{unionized}]_{water})}. Because log D is pH dependent, one must specify the pH at which the log D is measured. Of particular interest is the log D at pH=7.4 (the physiological pH of blood serum). For unionizable compounds, log P=log D at any pH. A higher value for log P or Log D indicates a more hydrophobic drug.

The phrase “substantially free of,” as used herein, means less than about 5 percent by weight, preferably less than about 2 percent by weight, more preferably less than about 1 percent by weight, even more preferably less than about 0.5 percent by weight, and most preferably less than about 0.2 percent by weight. For example, the phrase “a pharmaceutically acceptable organic solvent substantially free of a water” means that the amount of water is less than about 5 percent by weight, preferably less than about 2 percent by weight, more preferably less than about 1 percent by weight, even more preferably less than about 0.5 percent by weight, and most preferably less than about 0.2 percent by weight of the pharmaceutically acceptable organic solvent.

The term “about,” as used herein, to describe a range of values, applies to both the upper limit and the lower limit of the range. For example, the phrase “ranges from about 90:10 to 10:90” has the same meaning as “ranges from about 90:10 to about10:90.”

### The Dibasic Amino Acid Ester

The dibasic amino acid ester can be the ester of any dibasic amino acid, wherein the carboxylic acid group of the dibasic amino acid is esterified with a C₁-C₂₂ alcohol. Accordingly, the dibasic amino acid esters have the general formula (I):

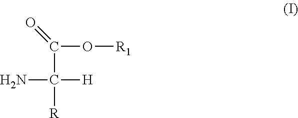

wherein

R is the amino acid side chain and includes a basic amine group; and

R₁ is a C₁ to C₂₂ hydrocarbon group.

As one of ordinary skill in the art would readily know, a wide variety of groups are possible for the amino acid side, R. For example, the amino acid side can be a hydrocarbon group that is substituted with an amine group. In one embodiment, R is a C₁-C₁₅ hydrocarbon group that is substituted with an amine group. In one embodiment, R is a C₁-C₁₀ hydrocarbon group that is substituted with an amine group. The hydrocarbon group may be further substituted with one or more other functional groups including, but not limited to, halo, nitro, cyano, thiol, hydroxy, aromatic group, and aromatic or non-aromatic heterocyclic group. The amino acid side chain, R, can also be, for example, —C₆H₄-amine group. The amine group can be —NH₂, NHRₐ, or —NHRₐR_{b}, wherein Rₐ and R_{b} are a C₁-C₁₈ hydrocarbon group. Rₐ and R_{b} can be connected to form a cyclic structure.

In one embodiment, the amine group is —NH₂.

The dibasic amino acid ester can be an ester of a naturally occurring dibasic amino acid or a synthetically prepared dibasic amino acid. The amino acid can be a D-amino acid or an L-amino acid. Preferably, the amino acid ester is the ester of a naturally occurring dibasic amino acid, for example, lysine, tryptophan, arginine, or histidine.

In one embodiment, the dibasic amino acid ester is an ester of lysine.

The hydrocarbon group, R₁, can be any C₁ to C₂₂ hydrocarbon group. Representative C₁ to C₂₂ hydrocarbon groups include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, allyl, cyclopentyl, cyclohexyl, cis-9-hexadecenyl, cis-9-octadecenyl, cis, cis-9, 12-octadecenyl, and cis, cis, cis-9, 12, 15-octadecatrienyl.

In one embodiment, R₁ is a straight or branched chain, saturated or unsaturated alkyl group.

In one embodiment, R₁ is a straight chain alkyl group.

In one embodiment, R₁ is a branched chain alkyl group.

In one embodiment, R₁ is a saturated alkyl group.

In one embodiment, R₁ is an unsaturated alkyl group.

In one embodiment, R₁ is a straight chain, saturated alkyl group.

In one embodiment, R₁ is a straight chain, unsaturated alkyl group.

In one embodiment, R₁ is a C₁-C₆ hydrocarbon group.

In one embodiment, R₁ is a C₁-C₆ straight chain alkyl group.

In one embodiment, R₁ is a C₁-C₆ branched chain alkyl group.

In one embodiment, R₁ is a C₁-C₆ saturated alkyl group.

In one embodiment, R₁ is a C₁-C₆ unsaturated alkyl group.

In one embodiment, R₁ is a C₆-C₁₈ hydrocarbon group.

In one embodiment, R₁ is a C₈-C₁₈ hydrocarbon group.

In one embodiment, R₁ is a C₁₀-C₁₈ hydrocarbon group.

In one embodiment, R₁ is a C₆-C₁₈ straight chain alkyl group.

In one embodiment, R₁ is a C₆-C₁₈ branched chain alkyl group.

In one embodiment, R₁ is a C₆-C₁₈ saturated alkyl group.

In one embodiment, R₁ is a C₆-C₁₈ unsaturated alkyl group.

In one embodiment, R₁ is a C₈-C₁₈ straight chain alkyl group.

In one embodiment, R₁ is a C₈-C₁₈ branched chain alkyl group.

In one embodiment, R₁ is a C₈-C₁₈ saturated alkyl group.

In one embodiment, R₁ is a C₈-C₁₈ unsaturated alkyl group.

In one embodiment, R₁ is a C₁₀-C₁₈ straight chain alkyl group.

In one embodiment, R₁ is a C₁₀-C₁₈ branched chain alkyl group.

In one embodiment, R₁ is a C₁₀-C₁₈ saturated alkyl group.

In one embodiment, R₁ is a C₁₀-C₁₈ unsaturated alkyl group.

In one embodiment, the group —OR₁ of the dibasic amino acid ester is a residue of a vitamin.

The vitamin can be any vitamin that includes a hydroxyl group. Illustrative vitamins include, but are not limited to, vitamin A (retinol), vitamin B₁ (thiamin), vitamin B₂ (riboflavin), vitamin B₅ (pantothenic acid), vitamin B₆, vitamin B₁₂ (cyanocobalamin), vitamin C, vitamin D, and vitamin E.

In one embodiment, the vitamin is vitamin A.

In one embodiment, the vitamin is vitamin B₁.

In one embodiment, the vitamin is vitamin B₂.

In one embodiment, the vitamin is vitamin B₅.

In one embodiment, the vitamin is vitamin B₆.

In one embodiment, the vitamin is vitamin B₁₂.

In one embodiment, the vitamin is vitamin C.

In one embodiment, the vitamin is vitamin D.

In one embodiment, the vitamin is vitamin E.

The dibasic amino acid esters can be obtained by esterifying a dibasic amino acid with an alcohol of formula R₁—OH, wherein R₁ is a C₁-C₂₂ alcohol (or a vitamin) using methods well known to those skilled in the art such as those described in J. March, Advanced Organic Chemistry, Reaction Mechanisms and Structure, 4^{th} ed. John Wiley & Sons, NY, 1992, pp. 393-400. The dibasic amino acids and alcohols (vitamins) of formula R₁—OH are commercially available or can be prepared by methods well known to those skilled in the art. When esterifying the dibasic amino acid with the alcohol (vitamin) of formula R₁—OH, it may be necessary to protect some other functional group of the dibasic amino acid, the alcohol, or the vitamin with a protecting group that is subsequently removed after the esterification reaction. One of ordinary skill in the art would readily know what functional groups would need to be protected before esterifying the dibasic amino acid with the alcohol (vitamin) of formula R₁—OH. Suitable protecting groups are known to those skilled in the art such as those described in T. W. Greene, et al. Protective Groups in Organic Synthesis, 3^{rd} ed. (1999). Methods for making amino acid esters are described in US 2006/0093632, the contents of which are incorporated herein by reference. Methods for making amino acid vitamin esters are described in U.S. Published Patent Application No. 2009/0023678, the contents of which are incorporated herein by reference.

### The Dibasic Amino Acid Amide

The dibasic amino acid amides can be an amide of any dibasic amino acid wherein the carboxylic acid group of the dibasic amino acid is reacted with a C₁-C₄₄ amine to provide an amide. Accordingly, the amino acid amides have the general formula (II):

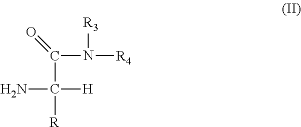

wherein

R is the amino acid side chain and includes a basic amine group;

R₃ is a C₁ to C₂₂ hydrocarbon group; and

R₄ is hydrogen or a C₁ to C₂₂ hydrocarbon group.

As one of ordinary skill in the art would readily know, a wide variety of groups are possible for the amino acid side, R. For example, R can be a hydrocarbon group that is substituted with an amine group. In one embodiment, R is a C₁-C₁₅ hydrocarbon group that is substituted with an amine group. In one embodiment R is a C₁-C₁₀ hydrocarbon group that is substituted with an amine group. The hydrocarbon group may be further substituted with one or more other functional groups including, but not limited to halo, nitro, cyano, thiol, hydroxy, aromatic group, and aromatic or non-aromatic heterocyclic group. The amino acid side chain, R, can also be, for example, —C₆H₄-amine group. The amine group can be —NH₂, NHRₐ, or —NHRₐR_{b}, wherein Rₐ and R_{b} are a C₁-C₁₈ hydrocarbon group. Rₐ and R_{b} can be connected to form a cyclic structure.

In one embodiment, the amine group is —NH₂.

The dibasic amino acid amide can be an amide of a naturally occurring dibasic amino acid or a synthetically prepared dibasic amino acid. The amino acid can be a D-amino acid or an L-amino acid. Preferably, the amino acid amide is the amide of a naturally occurring amino acid, for example, lysine, tryptophan, arginine, or histidine.

In one embodiment, the dibasic amino acid amide is an amide of lysine.

The R₃ group can be any C₁ to C₂₂ hydrocarbon group. The R₄ group can be hydrogen or any C₁ to C₂₂ hydrocarbon group. Representative C₁ to C₂₂ hydrocarbon groups include, but are not limited to, methyl, ethyl, propyl, butyl, pentyl, hexyl, heptyl, octyl, nonyl, decyl, undecyl, dodecyl, tridecyl, tetradecyl, pentadecyl, hexadecyl, heptadecyl, octadecyl, allyl, cyclopentyl, cyclohexyl, cis-9-hexadecenyl, cis-9-octadecenyl, cis, cis-9, 12-octadecenyl, and cis, cis, cis-9, 12, 15-octadecatrienyl.

In one embodiment, R₄ is hydrogen and R₃ is a straight or branched chain, saturated or unsaturated alkyl group.

In one embodiment, R₄ is hydrogen and R₃ is a straight chain alkyl group.

In one embodiment, R₄ is hydrogen and R₃ is a branched chain alkyl group.

In one embodiment, R₄ is hydrogen and R₃ is a saturated alkyl group.

In one embodiment, R₄ is hydrogen and R₃ is an unsaturated alkyl group.

In one embodiment, R₄ is hydrogen and R₃ is a straight chain, saturated alkyl group.

In one embodiment, R₄ is hydrogen and R₃ is a straight chain, unsaturated alkyl group.

In one embodiment, R₄ is hydrogen and R₃ is a C₁-C₆ hydrocarbon group.

In one embodiment, R₄ is hydrogen and R₃ is a C₁-C₆ straight chain alkyl group.

In one embodiment, R₄ is hydrogen and R₃ is a C₁-C₆ branched chain alkyl group.

In one embodiment, R₄ is hydrogen and R₃ is a C₁-C₆ saturated alkyl group.

In one embodiment, R₄ is hydrogen and R₃ is an C₁-C₆ unsaturated alkyl group.

In one embodiment, R₄ is hydrogen and R₃ is a C₆-C₁₈ hydrocarbon group.

In one embodiment, R₄ is hydrogen and R₃ is a C₆-C₁₈ straight chain alkyl group.

In one embodiment, R₄ is hydrogen and R₃ is a C₆-C₁₈ branched chain alkyl group.

In one embodiment, R₄ is hydrogen and R₃ is a C₆-C₁₈ saturated alkyl group.

In one embodiment, R₄ is hydrogen and R₃ is an C₆-C₁₈ unsaturated alkyl group.

In one embodiment, R₄ is hydrogen and R₃ is a C₈-C₁₈ hydrocarbon group.

In one embodiment, R₄ is hydrogen and R₃ is a C₈-C₁₈ straight chain alkyl group.

In one embodiment, R₄ is hydrogen and R₃ is a C₈-C₁₈ branched chain alkyl group.

In one embodiment, R₄ is hydrogen and R₃ is a C₈-C₁₈ saturated alkyl group.

In one embodiment, R₄ is hydrogen and R₃ is an C₈-C₁₈ unsaturated alkyl group.

In one embodiment, R₄ is hydrogen and R₃ is a C₁₀-C₁₈ hydrocarbon group.

In one embodiment, R₄ is hydrogen and R₃ is a C₁₀-C₁₈ straight chain alkyl group.

In one embodiment, R₄ is hydrogen and R₃ is a C₁₀-C₁₈ branched chain alkyl group.

In one embodiment, R₄ is hydrogen and R₃ is a C₁₀-C₁₈ saturated alkyl group.

In one embodiment, R₄ is hydrogen and R₃ is an C₁₀-C₁₈ unsaturated alkyl group.

In one embodiment, each of R₃ and R₄ are a straight or branched chain, saturated or unsaturated alkyl group, wherein R₃ and R₄ may be the same or different.

In one embodiment, each of R₃ and R₄ are a C₆-C₁₈ hydrocarbon group, wherein R₃ and R₄ may be the same or different.

In one embodiment, each of R₃ and R₄ are a C₈-C₁₈ hydrocarbon group, wherein R₃ and R₄ may be the same or different.

In one embodiment, each of R₃ and R₄ are a C₁₀-C₁₈ hydrocarbon group, wherein R₃ and R₄ may be the same or different.

In one embodiment, the combined number of carbon atoms in R₃ and R₄ is at least 6. In one embodiment, the combined number of carbon atoms in R₃ and R₄ is at least 8. In one embodiment, the combined number of carbon atoms in R₃ and R₄ is at least 10. In one embodiment, the combined number of carbon atoms in R₃ and R₄ is at least 12.

In one embodiment, the combined number of carbon atoms in R₃ and R₄ ranges from about 6 to 30. In one embodiment, the combined number of carbon atoms in R₃ and R₄ ranges from about 8 to 30. In one embodiment, the combined number of carbon atoms in R₃ and R₄ ranges from about 10 to 30. In one embodiment, the combined number of carbon atoms in R₃ and R₄ ranges from about 12 to 30. In one embodiment, the combined number of carbon atoms in R₃ and R₄ ranges from about 6 to 22. In one embodiment, the combined number of carbon atoms in R₃ and R₄ ranges from about 8 to 22. In one embodiment, the combined number of carbon atoms in R₃ and R₄ ranges from about 10 to 22. In one embodiment, the combined number of carbon atoms in R₃ and R₄ ranges from about 12 to 22. In one embodiment, the combined number of carbon atoms in R₃ and R₄ ranges from about 6 to 18. In one embodiment, the combined number of carbon atoms in R₃ and R₄ ranges from about 8 to 18. In one embodiment, the combined number of carbon atoms in R₃ and R₄ ranges from about 10 to 18. In one embodiment, the combined number of carbon atoms in R₃ and R₄ ranges from about 12 to 18.

The invention also encompasses dibasic amino acid amides of formula (II) wherein R₃ and R₄ are both hydrogen.

The amino acid amides can be obtained by converting the carboxylic acid group of the dibasic amino acid to an amide group using methods well known to those skilled in the art such as those described in J. March, Advanced Organic Chemistry, Reaction Mechanisms and Structure, 4^{th} ed. John Wiley & Sons, NY, 1992, pp. 417-427. Typically, the dibasic amino acid is converted to an amino acid derivative, such as an amino acid ester or an acid chloride of the dibasic amino acid, and the amino acid derivative is then reacted with an amine of formula NHR₃R₄ to provide the amino acid amide. The dibasic amino acids and amines of formula NHR₃R₄ are commercially available or can be prepared by methods well known to those skilled in the art. When forming the derivative of the dibasic amino acid or reacting the amino acid derivative with an amine of formula NHR₃R₄ it may be necessary to protect some other functional group of the amino acid derivative or the amine with a protecting group that is subsequently removed after the amidation reaction. One of ordinary skill in the art would readily know what functional groups would need to be protected before reacting the derivative of the amino acid with the amine of formula NHR₃R₄. Suitable protecting groups are known to those skilled in the art such as those described in T. W. Greene, et al. Protective Groups in Organic Synthesis, 3^{rd} ed. (1999).

### The Dicarboxylic Acid

Any dicarboxylic acid can be used. Suitable dicarboxylic acids include, but are not limited to, oxalic acid, malonic aid, succinic acid, glutamic acid, adipic acid, pimelic acid, and phthalic acid (in particular, 1,4-phthalic acid).

In one embodiment, the dicarboxylic acid is an N-acylated amino acid, wherein the side chain of the amino acid contains an acidic functional group. Thus, the N-acyl amino acids have the following general formula (III):

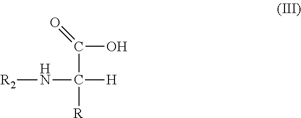

wherein

R is the amino acid side chain and includes an acidic functional group; and

R₂ is an acyl group of formula —C(O)—R₅, wherein R₅ is a substituted C₁ to C₂₁ hydrocarbon group, i.e., the acyl group is a C₁- to C₂₂ acyl group.

In another embodiment, R₂ is a residue of an acidic vitamin. In one embodiment, the acidic vitamin is folic acid.

As one of ordinary skill in the art would readily know, a wide variety of groups are possible for the amino acid side, R. For example, R can be a hydrocarbon group that is substituted with an acidic functional group. In one embodiment, R is a C₁-C₁₅ hydrocarbon group that is substituted with an acidic functional group. In one embodiment, R is a C₁-C₁₀ hydrocarbon group that is substituted with an acidic functional group. The hydrocarbon group may be further substituted with one or more other functional groups including, but not limited to halo, nitro, cyano, thiol, hydroxy, aromatic group, and aromatic or non-aromatic heterocyclic group. The amino acid side chain, R, can also be, for example, —C₆H₄-(acidic functional group). Acidic functional groups include, but are not limited to, —COOH (i.e., carboxylic acid groups), —S(O)₂—OH (i.e., sulfonic acid groups), —OP(O)(OR)(OH), —O(P)(O)₂, —P(O)(OR)(OH), —(P)(OH)₂), —OP(O)(R)(OH), and —P(O)(R)(OH), wherein R is a hydrocarbon group or phenyl group that can optionally be substituted. Preferably, the acidic functional group is —COOH.

The dicarboxylic acid that is an N-acylated amino acid can be obtained from a naturally occurring amino acid (for example aspartic acid or glutamic acid) or a synthetically prepared amino acid. The amino acid can be a D-amino acid or an L-amino acid. Preferably, the dicarboxylic acid that is an N-acylated amino acid is obtained from a naturally occurring amino acid, for example, aspartic acid or glutamic acid.

Representative acyl groups of formula —C(O)—R₅ include, but are not limited to, acetyl, propionyl, butanoyl, hexanoyl, caproyl, heptoyl, octoyl, nonoyl, decoyl, undecoyl, dodecoyl, tridecoyl, tetradecoyl, pentadecoyl, hexadecoyl, heptadecoyl, octadecoyl, laurolyl, myristoyl, palmitoyl, stearoyl, palmioleoyl, oleoyl, linoleoyl, linolenoyl, and benzoyl.

In one embodiment, R₅ is a C₁-C₅ hydrocarbon group, i.e., the acyl group of formula —C(O)—R₅ is a C₂-C₆ acyl group.

In one embodiment, R₅ is a C₅-C₂₁ hydrocarbon group, i.e., the acyl group of formula —C(O)—R₅ is a C₆-C₂₂ acyl group.

In one embodiment, R₅ is a C₇-C₂₁ hydrocarbon group, i.e., the acyl group of formula —C(O)—R₅ is a C₈-C₂₂ acyl group.

In one embodiment, R₅ is a C₉-C₂₁ hydrocarbon group, i.e., the acyl group of formula —C(O)—R₅ is a C₁₀-C₂₂ acyl group.

In one embodiment, R₅ is a C₅-C₁₇ hydrocarbon group, i.e., the acyl group of formula —C(O)—R₅ is a C₆-C₁₈ acyl group.

In one embodiment, R₅ is a C₇-C₁₇ hydrocarbon group, i.e., the acyl group of formula —C(O)—R₅ is a C₈-C₁₈ acyl group.

In one embodiment, R₅ is a C₉-C₁₇ hydrocarbon group, i.e., the acyl group of formula —C(O)—R₅ is a C₁₀-C₁₈ acyl group.

In one embodiment, the acyl group of formula —C(O)—R₅ is obtained from a saturated or unsaturated fatty acid.

In one embodiment, the acyl group of formula —C(O)—R₅ is a caproyl, laurolyl, myristoyl, palmitoyl, stearoyl, palmioleoyl, oleoyl, linoleoyl, or linolenoyl group.

The N-acylated amino acids can be obtained by methods well known to those skilled in the art. For example, the N-acylated amino acids can be obtained by reacting an amino acid with an acid halide of formula T-C(O)—R₅, wherein T is a halide, preferably chloride, and R₅ is as defined above, using methods well known to those skilled in the art. When N-acylating the amino acid with the acid halide of formula T-C(O)—R₅, it may be necessary to protect some other functional group of the amino acid or the acid halide with a protecting group that is subsequently removed after the acylation reaction. One of ordinary skill in the art would readily know what functional groups would need to be protected before acylating the amino acid with the acid halide of formula T-C(O)—R₅. Suitable protecting groups are known to those skilled in the art such as those described in T. W. Greene, et al. Protective Groups in Organic Synthesis, 3^{rd} ed. (1999). An illustrative method for making an N-acyl amino acid is provided in US 2006/0093632, the contents of which are incorporated herein by reference. When R₂ is a residue of an acidic vitamin, the N-acylated amino acid is made by reacting an amino acid with the acid chloride of the acidic vitamin.

Acid halides can be obtained using methods well known to those skilled in the art such as those described in J. March, Advanced Organic Chemistry, Reaction Mechanisms and Structure, 4^{th} ed. John Wiley & Sons, NY, 1992, pp. 437-8. For example, acid halides can be prepared by reacting a carboxylic acid with thionyl chloride, bromide, or iodide. Acid chlorides and bromides can also be prepared by reacting a carboxylic acid with phosphorous trichloride or phosphorous tribromide, respectively. Acid chlorides can also be prepared by reacting a carboxylic acid with Ph₃P in carbon tetrachloride. Acid fluorides can be prepared by reacting a carboxylic acid with cyanuric fluoride.

### The Pharmaceutically Active Compound

Any pharmaceutically active compound can be used in the pharmaceutical compositions of the invention.

In one embodiment, the pharmaceutically active compound is a neutral pharmaceutically active compound. In one embodiment, the neutral pharmaceutically active compound is a zwitterion. In one embodiment, the neutral pharmaceutically active compound is not a zwitterion.

In one embodiment, the pharmaceutically active compound is an acidic pharmaceutically active compound. In one embodiment, the pharmaceutically active compound is a salt of an acidic pharmaceutically active compound.

In one embodiment, the pharmaceutically active compound is a basic pharmaceutically active compound. In one embodiment, the pharmaceutically active compound is a salt of a basic pharmaceutically active compound.

In one embodiment, the pharmaceutically active compound is a hydrophobic pharmaceutically active compound.

In one embodiment, the pharmaceutically active compound is a hydrophilic pharmaceutically active compound.

Illustrative examples of pharmaceutically active compounds useful in the composition and methods of the invention include, but are not limited to, α-adrenergic agonists, β-adrenergic agonists, α-adrenergic blockers, β-adrenergic blockers, aldose reductase inhibitors, anabolics, analgesics (narcotic and non-narcotic), androgens, anesthetics, anorexics, anthelmintics (e.g., cestode, nematode, onchocerca, schistosoma, and the like), anti-allergics, anti-ameboics, anti-androgens, anti-anginals, anti-arrhythmics, anti-arteriosclerotics, anti-arthritics, antibiotics and other antibacterials, anti-cholinergics, anti-convulsants, anti-depressants, anti-diabetics agents, anti-diarrheals, anti-diuretics, anti-estrogens, antifungals, anti-glaucomas, anti-gonadotropins, anti-gout agents, anti-histaminics, anti-hyperlipoproteinemics, anti-hypertensives, anti-hyperthyroid agents, anti-hypertrophy agents, anti-hypotensives, anti-hypothyroid agents, anti-inflammatories, anti-malarials, antimicrobials, anti-migraine agents, anti-nausea agents, anti-neoplastics, antioxidants, antiparasitic agents, anti-parkinsonian agents, anti-pheochromocytoma agents, anti-pneumocytis agents, antiproliferative agents, anti-protozoals (e.g., leishmania, trichomonas, trypansoma, and the like), anti-pruritic agents, anti-psoratic agents, anti-psychotic agents, anti-pyretics, anti-rheumatics, anti ricketts agents, anti-seborrheic agents, antiseptics, anti-spasmodic agents, anti-thrombotic agents, antitussives, anti-ulcer agents, anti-urolithic agents, anti-venins, antivirals, anxiolytics, benzodiazepine antagonists, bronchodilators, calcium channel blockers, calcium regulators, cardiotonics, chelating agents, chemotherapeutics, cholecystokinin antagonists, cholelitholytic agents, choleretics, cholinergics, cholinesterase inhibitors, cholinesterase reactivators, central nervous system stimulants and agents, decongestants, diuretics, dopamine receptor agonists, drugs for treating or preventing pain, ectoparasiticides, enzymes, enzyme inducers, estrogens, gastric secretion inhibitors, glucocorticoids, gonad-stimulating principles, gonadotropic hormones, growth hormones, growth hormone releasing factors, growth stimulants, hemolytics, heparin agonists, hepatoprotectants, hypnotics, immune system boosters, immunomodulators, immunosuppressants, lactation stimulating hormones, LH-RH stimulating agonists, lipotropics, lupus erythmatosus suppressants, mineral corticoids, miotics, monoamine oxidase inhibitors, mucolytics, muscle relaxants, narcotic antagonists, neuroprotectives, neotropics, ovarian hormones, oxytocics, pepsin inhibitors, peristaltic stimulators, progestrogens, prolactin inhibitors, protoglandins, prostoglandin analogs, protease inhibitors, respiratory stimulants, sclerosing agents, sedatives, steroids, thrombolytics, thyrotropic hormones, transdermal penetration enhancers, uricosurics, vasoconstrictors, vasodilators (e.g., cerebral, coronary, peropheral, and the like), vasoprotectants, vitamins, vitamin source extracts, vulneraries (including, but not limited to, those listed in U.S. Patent No. 5,719,197, the disclosure of which is incorporated herein by reference), and combinations thereof. Other additionally or alternately acceptable pharmaceutically active agents can be found, e.g., in U.S. Patent No. 6,221,383, the disclosure of which is incorporated herein by reference.

In one embodiment, the pharmaceutically active compound is an antibacterial agent. Examples of useful antibacterial agents include, but are not limited to, β-lactam antibiotics such as penicillins, amoxicillin, ampicillin, and cephalosporins; macrolide antibiotics such as oleandomycin and erythromycin; tetracyclines such as tetracycline, oxytetracycline, and chlortetracycline; procaine penicillin G; quinolones such as nalidixic acid and norfloxacin; sulfonamides; chloramphenicol; florfenicol; thiamphenicol, aminoglycosides such as streptomycin, kanamycin, and gentamycins; nucleoside antibiotics such as polyoxin B; actinorhodine; bacitracin; candicidin A; ceftiofor; clindamycin; cycloheximide; cycloserine; fosfomycin; griseofulvin; metronidazole; monensin; novobiocin; rifampin; streptothricin; tetranactin; tilmicosin; tylosin; actinomycin D; adriamycin; bleomycin B2; glycolipids such as moenomycin A; mitomycin C; nojirimycin; valinomycin; and vancomycin; (See, e.g., Bradford P. Smith, Large Animal Internal Medicine, 2^{nd} ed., Mosby, St. Louis, 1996, p. 644, and S. Birchard and R. Sherding, Saunders Manual of Small Animal Practice, W. B. Saunders Company, Philadelphia, 1994, p. 739).

In one embodiment, the pharmaceutically active compound is an antifungal agent. Examples of useful antifungal agents include, but are not limited to terbinafine, amphotericin B, ketaconazole, miconazole, 5-fluorocytosine, enilconazole, itraconazole, thiabendazole, and iodides (See, e.g., Bradford P. Smith, Large Animal Internal Medicine, 2^{nd} ed., Mosby, St. Louis, 1996, p. 576, and S. Birchard and R. Sherding, Saunders Manual of Small Animal Practice, W. B. Saunders Company, Philadelphia, 1994, p. 576).

In one embodiment, the pharmaceutically active compound is an antiviral agent. Examples of useful antiviral agents include, but are not limited to, interferon and adefovir (See, e.g., Bradford P. Smith, Large Animal Internal Medicine, 2^{nd} ed., Mosby, St. Louis, 1996, p. 646).

In one embodiment, the pharmaceutically active compound is an antiparasitic agent. Examples of useful antiparasitic agents include, but are not limited to, benzimidazoles, such as thiabendazole, fenbendazole, mebendazole, oxfendazole, oxibendazole, albendazole, parbendazole, and febantel; tetrahydropyridines such as morantel tartrate/pyrantel pamoate; levamisole, organophosphates such as haloxon, coumaphos, trichlorfon, and dichlorvos; piperazine salts; ivermectin; and phenothiazine (See, e.g., Bradford P. Smith, Large Animal Internal Medicine, 2^{nd} ed., Mosby, St. Louis, 1996, p. 1688).

In one embodiment, the pharmaceutically active compound is an anti-inflammatory agent. Examples of useful antiinflammatory agents include, but are not limited to, steroids such as betamethazone; corticosteroids such as dexamethasone; antihistamines; and non-steroidal antiinflammatory drugs such as aspirin, flunixin meglumine, phenylbutazone, diclofenac, naproxen, ketoprofen, carprofen, and ibuprofin (See, e.g., Bradford P. Smith, Large Animal Internal Medicine, 2nd edn., Mosby, St. Louis, 1996, p. 645).

In one embodiment, the pharmaceutically active compound is a hydrophobic pharmaceutically active compound.

In one embodiment, the pharmaceutically active compound has a log P_{oct/water} value of at least about 1. In one embodiment, the pharmaceutically active compound has a log P_{oct/water} value of at least about 1.5. In one embodiment, the pharmaceutically active compound has a log P_{oct/water} value of at least about 2. In one embodiment, the pharmaceutically active compound has a log P_{oct/water} value of at least about 2.5.

In one embodiment, the pharmaceutically active compound has a log D_{oct/water} value at pH 7.4 of at least about 1. In one embodiment, the pharmaceutically active compound has a log D_{oct/water} value at pH 7.4 of at least about 1.5. In one embodiment, the pharmaceutically active compound has a log D_{oct/water} value at pH 7.4 of at least about 2. In one embodiment, the pharmaceutically active compound has a log D_{oct/water} value at pH 7.4 of at least about 2.5.

In one embodiment, the pharmaceutically active compound is a hydrophilic pharmaceutically active compound.

In one embodiment, the pharmaceutically active compound has a log P_{oct/water} value of less than about 1. In one embodiment, the pharmaceutically active compound has a log P_{oct/water} value of less than about 0.5. In one embodiment, the pharmaceutically active compound has a log P_{oct/water} value of less than about 0.25. In one embodiment, the pharmaceutically active compound has a log P_{oct/water} value of less than about 0.1.

In one embodiment, the pharmaceutically active compound has a log D_{oct/water} value at pH 7.4 of less than about 1. In one embodiment, the pharmaceutically active compound has a log D_{oct/water} value at pH 7.4 of less than about 0.5. In one embodiment, the pharmaceutically active compound has a log D_{oct/water} value at pH 7.4 of less than about 0.25. In one embodiment, the pharmaceutically active compound has a log D_{oct/water} value at pH 7.4 of less than about 0.1.

### The Pharmaceutical Compositions

In one embodiment, the pharmaceutical compositions comprise:

(i) a dibasic amino acid ester or dibasic amino acid amide;

(ii) a dicarboxylic acid;

(iii) a pharmaceutically active compound or pharmaceutically acceptable salt thereof; and

(iv) a pharmaceutically acceptable organic solvent.

In one embodiment, the pharmaceutical composition comprises (i) a dibasic amino acid ester; (ii) a dicarboxylic acid; (iii) a pharmaceutically active compound or pharmaceutically acceptable salt thereof; and (iv) a pharmaceutically acceptable organic solvent. In one embodiment, the dicarboxylic acid is a N-acylated amino acid, wherein the side chain of the amino acid contains an acidic functional group.

In one embodiment, the dibasic amino acid ester is an dibasic amino acid vitamin ester.

In one embodiment, the dibasic amino acid ester is an dibasic amino acid vitamin ester and the dicarboxylic acid is an N-acylated amino acid, wherein the side chain of the amino acid contains an acidic functional group.

In one embodiment, the pharmaceutical compositions comprises (i) a dibasic amino acid amide; (ii) a dicarboxylic acid; (iii) a pharmaceutically active compound or pharmaceutically acceptable salt thereof; and (iv) a pharmaceutically acceptable organic solvent. In one embodiment, the dicarboxylic acid is a N-acylated amino acid, wherein the side chain of the amino acid contains an acidic functional group.

The molar ratio of the dibasic amino acid ester or dibasic amino acid amide to the dicarboxylic acid typically ranges from about 1.5:1 to 1:1.5, preferably from about 1.25:1 to about 1:1.25, more preferably from about 1.1:1 to 1:1.1, and most preferably about 1:1. Other ratios, however, are within the scope of the invention.

The amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid typically ranges from about 0.2 to 10 percent. In one embodiment, the amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid ranges from about 1 to 5 percent. In one embodiment, the amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid ranges from about 0.5 to 1 percent.

In one embodiment, the combined amount of the dibasic amino acid ester or dibasic amino acid amide and the dicarboxylic acid is about 2 to 75 times more by weight than the amount of the pharmaceutically active compound. In one embodiment, the combined amount of the dibasic amino acid ester or dibasic amino acid amide and the dicarboxylic acid is about 10 to 50 times more by weight than the amount of the pharmaceutically active compound. In one embodiment, the combined amount of the dibasic amino acid ester or dibasic amino acid amide and the dicarboxylic acid is about 20 to 40 times more by weight than the amount of the pharmaceutically active compound.

In the pharmaceutical composition comprising a pharmaceutically acceptable organic solvent the combined amount of the pharmaceutically active compound or pharmaceutically acceptable salt thereof, the dibasic amino acid ester or dibasic amino acid amide, and the dicarboxylic acid typically ranges from about 1 to 50 percent by weight percent of the composition. In one embodiment, the combined amount of the pharmaceutically active compound or pharmaceutically acceptable salt thereof, the dibasic amino acid ester or dibasic amino acid amide, and the dicarboxylic acid typically ranges from about 1.5 to 40 percent by weight percent of the composition. In one embodiment, the combined amount of the pharmaceutically active compound or pharmaceutically acceptable salt thereof, the dibasic amino acid ester or dibasic amino acid amide, and the dicarboxylic acid typically ranges from about 2 to 30 percent by weight percent of the composition.

One of ordinary skill in the art, however, will recognize that the combined amount of the pharmaceutically active compound or pharmaceutically acceptable salt thereof, the dibasic amino acid ester or dibasic amino acid amide, and the dicarboxylic acid can vary widely depending on the pharmaceutically active compound or pharmaceutically acceptable salt thereof, the dibasic amino acid ester or dibasic amino acid amide, and the dicarboxylic acid, and the solvent used in the pharmaceutical composition. Similarly, the ratio of the individual components can vary widely.

Any pharmaceutically acceptable organic solvent can be used in the pharmaceutical compositions of the invention. Representative, pharmaceutically acceptable organic solvents include, but are not limited to, pyrrolidone, N-methyl-2-pyrrolidone, polyethylene glycol, propylene glycol (i.e., 1,3-propylene glycol, 1,2-propylene glycol, or a mixture thereof), glycerol formal, isosorbid dimethyl ether, ethanol, dimethyl sulfoxide, tetraglycol, tetrahydrofurfuryl alcohol, triacetin, propylene carbonate, dimethyl acetamide, dimethyl formamide, dimethyl sulfoxide, and combinations thereof.

In one embodiment, the pharmaceutically acceptable organic solvent is a water soluble solvent. A representative pharmaceutically acceptable water soluble organic solvent is triacetin.

In one embodiment, the pharmaceutically acceptable organic solvent is a water miscible solvent. Representative pharmaceutically acceptable water miscible organic solvents include, but are not limited to, glycerol formal, polyethylene glycol, and propylene glycol.

In one embodiment, the pharmaceutically acceptable organic solvent is a solvent that is recognized as GRAS by the FDA for administration or consumption by animals.

In one embodiment, the pharmaceutically acceptable organic solvent is a solvent that is recognized as GRAS by the FDA for administration or consumption by humans.

In one embodiment, the pharmaceutically acceptable organic solvent is substantially free of water. Pharmaceutically acceptable organic solvents that are substantially free of water are advantageous since they are not conducive to bacterial growth. Accordingly, it is typically not necessary to include a preservative in pharmaceutical compositions that are substantially free of water.

In one embodiment, the pharmaceutical composition is injectable.

In one embodiment, the pharmaceutical composition is injectable and forms a precipitate when injected into water.

Although, the pharmaceutical composition forms a precipitate when it is injected into water, if the resulting aqueous mixture containing the precipitate is subjected to mechanical forces (e.g., shaking, stirring, or sonication) micelles or liposomes will typically form.

The micelles can be characterized by their particle size. Particle size can be determined by, for example, Dynamic Light Scattering (Photon Correlation Spectroscopy or Quasi-Elastic Light Scattering). The particle size can range from about 1 nm to about 1000 nm. In one embodiment, the particle size ranges from about 20 nm to about 500 nm. In one embodiment, the particle size ranges from about 50 nm to about 300 nm. In one embodiment, the particle size ranges from about 1000 nm to about 300 nm.

The micelles can also be characterized by their zeta potential. Zeta potential can be determined using, for example, a Malvern Zetasizer Nano ZS instrument (commercially available from Malvern Instrumentsof Westborough, Mass.), which can also determine particle size. The zeta potential can range from about −20 mv to +20 mv. In one embodiment, the zeta potential ranges from about −10 mv to +10 mv. In one embodiment, the zeta potential ranges from about −5 mv to +5 mv. In one embodiment, the zeta potential ranges from about -10 mv to 0 mv. In one embodiment, the zeta potential ranges from about 0 mv to +10 mv. In one embodiment, the zeta potential is about 0 mv.

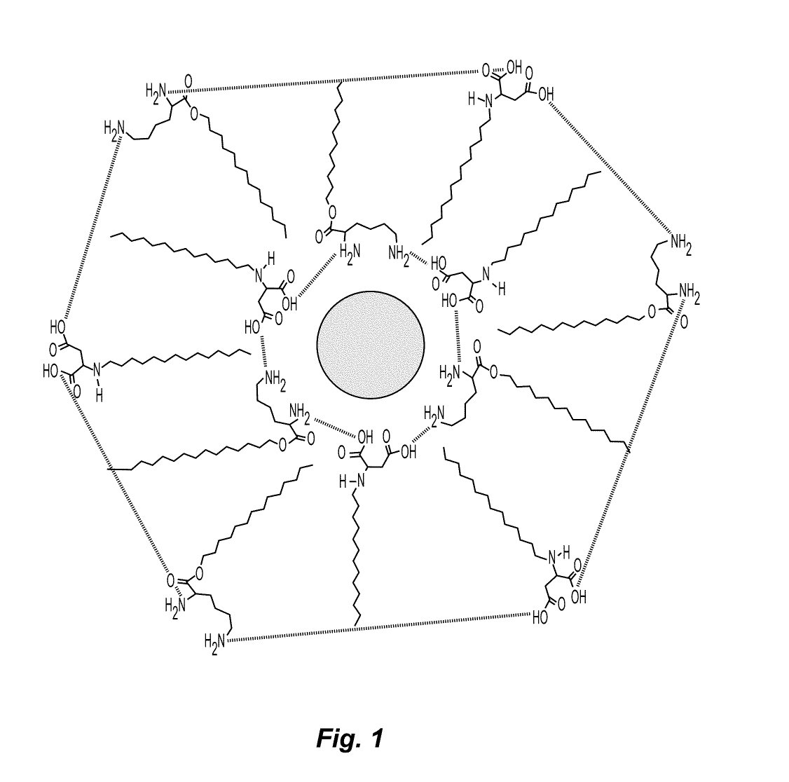

The precipitate can take the form of a solid, a crystal, a gummy mass, or a gel. Without wishing to be bound by theory, it is believed that the amine groups of the dibasic amino acid esters or dibasic amino acid amides are protonated by the acidic functional groups of the dicarboxylic acid to form an extended salt structure such as depicted below, using a lysine ester and a N-acylated glutamic acid as illustrative of the dibasic amino acid and the dicarboxylic acid, respectively:

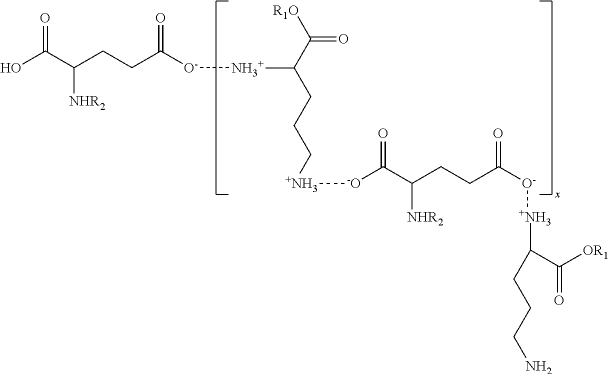

wherein x is an integer. The extended salt structure can be characterized as a non-covalent polymeric structure. Without wishing to be bound by theory it is believed that the extended salt structure is soluble in the pharmaceutically acceptable organic solvent but insoluble in water. Thus, the pharmaceutical composition forms a precipitate when injected into water. It is believed that the precipitate entraps the pharmaceutically active compound or pharmaceutically acceptable salt thereof and releases it slowly over time. FIG. 1 illustrates the pharmaceutically active compound or pharmaceutically acceptable salt thereof entrapped within the extended salt structure. It is believed that the micelles and liposomes, which form when the aqueous mixture containing the precipitate is subjected to mechanical forces, have a similar structure. FIG. 1, however, is only illustrative, and should not be construed as limiting the structure of precipitate. One will readily understand that when the pharmaceutically active compound is an acidic pharmaceutically active compound, the acidic pharmaceutically active compound will also protonate the dibasic amino acid ester or dibasic amino acid amide. Similarly, when the pharmaceutically active compound is a basic pharmaceutically active compound, the basic pharmaceutically active compound will also be protonated by the dicarboxylic acid.

Accordingly, when the pharmaceutical composition is injected into an animal, at least a portion of the pharmaceutical composition precipitates at the injection site to provide a drug depot. Without wishing to be bound by theory, it is believed that when the pharmaceutical compositions are injected into an animal, the pharmaceutically acceptable organic solvent diffuses away from the injection site and aqueous bodily fluids diffuse towards the injection site, resulting in an increase in concentration of water at the injection site, that causes at least a portion of the composition to precipitate and form a drug depot. The precipitate can take the form of a solid, a crystal, a gummy mass, or a gel. The precipitate, however, provides a depot of the pharmaceutically active compound or pharmaceutically acceptable salt thereof at the injection site that releases the pharmaceutically active compound over time.

In one embodiment, the pharmaceutical composition is injectable and forms a depot when administered to an animal by injection.

By varying the lipophilicity and/or molecular weight of the dibasic amino acid ester or dibasic amino acid amide it is possible to vary the rate at which the pharmaceutically active compound is released from the drug depot. Generally, the more lipophilic the dibasic amino acid ester or dibasic amino acid amide, the more slowly drug is released. The lipophilicity and/or molecular weight of the dibasic amino acid ester or dibasic amino acid amide can be varied by varying the amino acid and/or the R₁ group of the dibasic amino acid ester or the R₃ and R₄ groups of the amino acid amide. For example, when R is a hydrocarbon group, the higher the molecular weight of R, the more lipophilic is the dibasic amino acid ester or dibasic amino acid amide. Similarly, when R₁, R₃, and R₄ are a hydrocarbon group, the higher the molecular weight of R₁, R₃, and R₄, the more lipophilic is the dibasic amino acid ester or dibasic amino acid amide.

The rate at which the pharmaceutically active compound is released from the drug depot can also be varied by varying the lipophilicity and/or molecular weight of the dicarboxylic acid. Generally, the more lipophilic the carboxylic acid, the more slowly drug is released. The lipophilicity of the carboxylic acid can be varied by varying the molecular weight of the carboxylic acid. Generally, the higher the molecular weight of the carboxylic acid, the more slowly drug is released. For example, the lipophilicity and/or molecular weight of dicarboxylic acids that are N-acyl amino acids can be varied by varying the R₅ group of the acyl group.

A measure of how rapidly the pharmaceutically active compound or pharmaceutically acceptable salt thereof will be released from a drug depot can be estimated by the following illustrative in vitro method: An aliquot (about 1 mL) of the pharmaceutical compositions is sealed in a dialysis bag (commercially available from Pierce Biotechnology, Inc. of Rockford Ill.) and the dialysis bag suspended in a flask containing about 150 mL of phosphate buffered saline at pH 7.4. Aliquots of saline are then removed at various intervals and the concentration of the pharmaceutically active compound or pharmaceutically acceptable salt thereof determined using any analytical method known to those skilled in the art. Suitable analytical methods include, but are riot limited to, high pressure liquid chromatography (HPLC) and gas chromatography (GC). HPLC and GC are particularly useful analytical methods. When HPLC or GC is used as the analytical method, the concentration of the pharmaceutically active compound or pharmaceutically acceptable salt thereof is typically determined by comparing the area under the curve for the HPLC or GC peak corresponding to the pharmaceutically active compound or pharmaceutically acceptable salt thereof to a standard curve of peak areas v. known concentrations of the pharmaceutically active compound or pharmaceutically acceptable salt thereof in phosphate-buffered saline. The amount of pharmaceutically active compound or pharmaceutically acceptable salt thereof measured in the saline as a function of time is a measure of how rapidly the pharmaceutically active compound or pharmaceutically acceptable salt thereof will be released from a drug depot. In one embodiment, less than 20% of the pharmaceutically active compound is released in 6 hours. In one embodiment, less than 10% of the pharmaceutically active compound is released in 6 hours. In one embodiment, less than 5% of the pharmaceutically active compound is releases in 6 hours. In one embodiment, less than 20% of the pharmaceutically active compound is released in 8 hours. In one embodiment, less than 10% of the pharmaceutically active compound is released in 8 hours. In one embodiment, less than 5% of the pharmaceutically active compound is released in 8 hours. In one embodiment, less than 20% of the pharmaceutically active compound is released in 10 hours. In one embodiment, less than 10% of the pharmaceutically active compound is released in 10 hours. In one embodiment, less than 5% of the pharmaceutically active compound is released in 10 hours.

In one embodiment, the amount of pharmaceutically active compound released from the composition after a specified amount of time is less than the amount of pharmaceutically active compound that is released from a comparable composition that does not include the dibasic amino acid ester or dibasic amino acid amide or does not include the dicarboxylic acid during the same amount of time. In one embodiment, the amount of pharmaceutically active compound released from the composition after 6 hours is less than the amount of pharmaceutically active compound that is released from a comparable composition that does not include the dibasic amino acid ester or dibasic amino acid amide or does not include the dicarboxylic acid. In the comparable composition without the dicarboxylic acid, the dicarboxylic acid is replaced with an equivalent weight of the dibasic amino acid ester or dibasic amino acid amide or the dicarboxylic acid is replaced with an equivalent weight of an ester of the dicarboxylic acid made from a C₁-C₅ alcohol, preferably a C₁ alcohol. Similarly, in the comparable composition without the dibasic amino acid ester or dibasic amino acid amide, the dibasic amino acid ester or dibasic amino acid amide is replaced with an equivalent weight of the dicarboxylic acid. In one embodiment, the amount of pharmaceutically active compound released from the composition after 8 hours is less than the amount of pharmaceutically active compound that is released from a comparable composition that does not include the dibasic amino acid ester or dibasic amino acid amide or does not include the dicarboxylic acid. In one embodiment, the amount of pharmaceutically active compound released from the composition after 10 hours is less than the amount of pharmaceutically active compound that is released from a comparable composition that does not include the dibasic amino acid ester or dibasic amino acid amide or does not include the dicarboxylic acid.

A measure of how rapidly the pharmaceutically active compound or pharmaceutically acceptable salt thereof will be released from a drug depot in vivo can be determined by administering the pharmaceutical composition to an animal, removing blood samples from the animal as a function of time, and measuring the concentration of the pharmaceutically active compound or pharmaceutically acceptable salt thereof in the blood samples. The concentration of the pharmaceutically active compound or pharmaceutically acceptable salt thereof in the blood samples can be determined using methods well known to those skilled in the art.

The invention also encompasses the precipitate, i.e., a composition comprising a dibasic amino acid ester or dibasic amino acid amide, a dicarboxylic acid, and a pharmaceutically active compound. In one embodiment, the composition comprising a dibasic amino acid ester or dibasic amino acid amide, a dicarboxylic acid, and a pharmaceutically active compound is obtained by a process wherein a solution comprising a dibasic amino acid ester or dibasic amino acid amide, a dicarboxylic acid, a pharmaceutically active compound, and a water miscible organic solvent is added to water to provide a precipitate that is then collected, for example, by filtration. Optionally, the precipitate can be dried. In another embodiment, the precipitate is recovered by removing the solvent by lyophilization.

The precipitate can be formulated into a solid oral dosage form for administration to an animal. In one embodiment, the precipitate is formulated into a solid oral dosage form for oral administration to an animal.

In another embodiment, the pharmaceutical composition comprises micelles or liposomes suspended in an aqueous solvent, wherein the micelles or liposomes comprise
(i) the dibasic amino acid ester or dibasic amino acid amide;
(ii) the dicarboxylic acid; and
(iii) the pharmaceutically active compound or pharmaceutically acceptable salt thereof.

In one embodiment, the micelles or liposomes comprise (i) a dibasic amino acid ester; (ii) a dicarboxylic acid; and (iii) a pharmaceutically active compound or pharmaceutically acceptable salt thereof. In one embodiment, the dicarboxylic acid is a N-acylated amino acid, wherein the side chain of the amino acid contains an acidic functional group.

In one embodiment, the dibasic amino acid ester is an dibasic amino acid vitamin ester.

In one embodiment, the dibasic amino acid ester is an dibasic amino acid vitamin ester and the dicarboxylic acid is an N-acylated amino acid, wherein the side chain of the amino acid contains an acidic functional group.

In one embodiment, the micelles or liposomes comprise (i) a dibasic amino acid amide; (ii) a dicarboxylic acid; and (iii) a pharmaceutically active compound or pharmaceutically acceptable salt thereof. In one embodiment, the dicarboxylic acid is a N-acylated amino acid, wherein the side chain of the amino acid contains an acidic functional group.

The molar ratio of the dibasic amino acid ester or dibasic amino acid amide to the dicarboxylic acid typically ranges from about 1.5:1 to 1:1.5, preferably from about 1.25:1 to about 1:1.25, more preferably from about 1.1:1 to 1:1.1, and most preferably about 1:1. Other ratios, however, are within the scope of the invention.

The amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid typically ranges from about 0.2 to 10 percent. In one embodiment, the amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid ranges from about 1 to 5 percent. In one embodiment, the amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid ranges from about 0.5 to 1 percent.

In one embodiment, the combined amount of the dibasic amino acid ester or dibasic amino acid amide and the dicarboxylic acid is about 2 to 75 times more by weight than the amount of the pharmaceutically active compound. In one embodiment, the combined amount of the dibasic amino acid ester or dibasic amino acid amide and the dicarboxylic acid is about 10 to 50 times more by weight than the amount of the pharmaceutically active compound. In one embodiment, the combined amount of the dibasic amino acid ester or dibasic amino acid amide and the dicarboxylic acid is about 20 to 40 times more by weight than the amount of the pharmaceutically active compound.

In the micelle or liposome containing pharmaceutical composition the combined amount of the pharmaceutically active compound or pharmaceutically acceptable salt thereof, the dibasic amino acid ester or dibasic amino acid amide, and the dicarboxylic acid typically ranges from about 1 to 50 percent by weight percent of the composition. In one embodiment, the combined amount of the pharmaceutically active compound or pharmaceutically acceptable salt thereof, the dibasic amino acid ester or dibasic amino acid amide, and the dicarboxylic acid ranges from about 1.5 to 40 percent by weight percent of the composition. In one embodiment, the combined amount of the pharmaceutically active compound or pharmaceutically acceptable salt thereof, the dibasic amino acid ester or dibasic amino acid amide, and the dicarboxylic acid ranges from about 2 to 30 percent by weight percent of the composition.

One of ordinary skill in the art, however, will recognize that the combined amount of the pharmaceutically active compound or pharmaceutically acceptable salt thereof, the dibasic amino acid ester or dibasic amino acid amide, and the dicarboxylic acid can vary widely depending on the pharmaceutically active compound or pharmaceutically acceptable salt thereof, the dibasic amino acid ester or dibasic amino acid amide, and the dicarboxylic acid.

The micelle containing and liposome containing pharmaceutical compositions can be administered to an animal by injection. The micelle containing and liposome containing pharmaceutical compositions, however, typically, do not form a depot when administered to an animal by injection. Rather, the micelle or liposome structures are maintained, at least initially, after administration, and then overtime break down to release the pharmaceutically active compound, thus, providing sustained release of the pharmaceutically active compound. Without wishing to be bound by theory, it is believed that the structure of the micelles and liposomes are similar to that of the precipitate depicted in FIG. 1.

The micelle containing and liposome containing pharmaceutical compositions can also be orally administered to an animal.

By varying the lipophilicity and/or molecular weight of the dibasic amino acid ester or dibasic amino acid amide and/or by varying the lipophilicity and/or molecular weight of the dicarboxylic acid, as discussed above, it is possible to vary the rate at which the pharmaceutically active compound is released from the micelles or liposomes.

A measure of how rapidly the pharmaceutically active compound or pharmaceutically acceptable salt thereof will be released from the micelles or liposomes can be estimated by using the same in vitro method that is used to determine how rapidly the pharmaceutically active compound or pharmaceutically acceptable salt thereof is released from a depot.

A measure of how rapidly the pharmaceutically active compound or pharmaceutically acceptable salt thereof will be released from the micelles or liposomes in vivo can be determined using a method similar to that used to determine how rapidly the pharmaceutically active compound or pharmaceutically acceptable salt thereof is released from a drug depot in vivo.

In one embodiment, the amount of pharmaceutically active compound released from the composition after a specified amount of time is less than the amount of pharmaceutically active compound that is released from a comparable composition that does not include the dibasic amino acid ester or dibasic amino acid amide or does not include the dicarboxylic acid during the same amount of time. In the comparable composition without the dicarboxylic acid, the dicarboxylic acid is replaced with an equivalent weight of the dibasic amino acid ester or dibasic amino acid amide or the dicarboxylic acid is replaced with an equivalent weight of an ester of the dicarboxylic acid made from a C₁-C₅ alcohol, preferably a C₁ alcohol. Similarly, in the comparable composition without the dibasic amino acid ester or dibasic amino acid amide, the dibasic amino acid ester or dibasic amino acid amide is replaced with an equivalent weight of the dicarboxylic acid. In one embodiment, the amount of pharmaceutically active compound released from the composition after 8 hours is less than the amount of pharmaceutically active compound that is released from a comparable composition that does not include the dibasic amino acid ester or dibasic amino acid amide or does not include the dicarboxylic acid. In one embodiment, the amount of pharmaceutically active compound released from the composition after 10 hours is less than the amount of pharmaceutically active compound that is released from a comparable composition that does not include the dibasic amino acid ester or dibasic amino acid amide or does not include the dicarboxylic acid.

When the composition includes a dibasic amino acid ester or dibasic amino acid amide, a dicarboxylic acid, and a basic pharmaceutically active compound, it is recognized that the dicarboxylic acid will also protonate some of the basic pharmaceutically active compound. Similarly, when the compositions include a dibasic amino acid ester or dibasic amino acid amide, a dicarboxylic acid, and an acidic pharmaceutically active compound, it is recognized that the dibasic amino acid ester or dibasic amino acid amide will also be protonated by the acidic pharmaceutically active compound. By selecting appropriate amounts of the dibasic amino acid ester or dibasic amino acid amide, the dicarboxylic acid, and the basic or acidic pharmaceutically active compound, precipitates or micelles or liposomes having the extended salt structure described above will still be formed even with basic and acidic pharmaceutically active compounds.

Also when the pharmaceutically active compound is a salt of a pharmaceutically active compound it is recognized that the anion (cation) of the salt of the pharmaceutically active compound will also form a salt with the dibasic amino acid ester or amino acid amide (dicarboxylic acid). For example, if the salt of a pharmaceutically active compound is a salt formed between a basic pharmaceutically active compound and acetic acid, the composition will include, in addition to the salt formed between the basic pharmaceutically active compound and acetic acid and the salt formed where the dicarboxylic acid protonates the dibasic amino acid ester or amino acid amide e., the extended salt structure), a salt wherein acetic acid protonates the dibasic amino acid ester or amino acid amide and a salt wherein the dicarboxylic acid protonates the basic pharmaceutically active compound. Any one or all of these species can be part of the precipitate that forms when the pharmaceutical composition is injected into an animal to form a drug depot that slowly releases the pharmaceutically active compound over time. By selecting appropriate amounts of the dibasic amino acid ester or dibasic amino acid amide, the dicarboxylic acid, and the basic or acidic pharmaceutically active compound, precipitates or forms micelles or liposomes having the extended salt structure described above will still be formed even with salts of basic or acidic pharmaceutically active compounds.

The pharmaceutical compositions of the invention can further include other excipients and/or additives routinely used in pharmaceutical compositions. For example, the pharmaceutical formulations may include a preservative to inhibit microbial growth. Suitable preservatives include, but are not limited to, parabens such as methyl, ethyl, and propyl parabens; chlorobutanol; sodium benzoate; myristyl-gamma-picolinium chloride; benzyl alcohol; and ethyl alcohol. Preservatives, when present, are typically present in an amount of about 5 mg to 250 mg per mL of pharmaceutical composition and preferably about 5 mg to 100 mg per mL of pharmaceutical composition.

In one embodiment, the injectable compositions include a local anesthetic such as lidocaine to lessen pain at the site of the injection.

### Manufacturing the Pharmaceutical Compositions

To prepare the pharmaceutical compositions of the invention comprising (i) a dibasic amino acid ester or dibasic amino acid amide, (ii) a dicarboxylic acid, (iii) a pharmaceutically active compound or pharmaceutically acceptable salt thereof, and (iv) a pharmaceutically acceptable organic solvent, the dibasic amino acid ester or dibasic amino acid amide, the dicarboxylic acid, and the pharmaceutically active compound are dissolved in the pharmaceutically acceptable organic solvent to provide a solution (typically about 90% of the amount of the solvent desired in the final pharmaceutical composition). Additional excipients and/or additives can then be dissolved in the solution. The order of addition is not important. Typically, the addition is performed with stirring. Additional pharmaceutically acceptable organic solvent is then added to provide the desired concentration of the dibasic amino acid ester or dibasic amino acid amide, the dicarboxylic acid, and the pharmaceutically active compound or pharmaceutically acceptable salt thereof. The solution of the dibasic amino acid ester or dibasic amino acid amide, the dicarboxylic acid, and the pharmaceutically active compound or pharmaceutically acceptable salt thereof, and additional excipients and/or additives can then be filtered, preferably sterile filtered, directly into bottles. In one embodiment, the pharmaceutically acceptable organic solvent is water miscible.

The pharmaceutical compositions comprising micelles or liposomes can be made by dissolving the dibasic amino acid ester or dibasic amino acid amide, the dicarboxylic acid, and the pharmaceutically active compound in a water miscible organic solvent to provide a solution; combining the resulting solution with water, with stirring; and then removing the organic solvent, typically by evaporation. In another embodiment, the solution of the dibasic amino acid ester or dibasic amino acid amide, the dicarboxylic acid, and the pharmaceutically active compound in an organic solvent is added to water, with stirring, to provide a mixture and the resulting mixture lyophilized to provide a powder. The resulting powder, when recombined with water, results in the pharmaceutical compositions comprising micelles or liposomes. The invention is also directed to the lyophilized powder. Addition with stirring can be accomplished, for example, by sonication, using a vortex mixer, or using a shear mixer.

The pharmaceutical compositions can be sterilized by sterile filtering or can be terminally sterilized using, for example, an autoclave.

The invention further relates to a method of manufacturing the pharmaceutical composition of the invention.

### Methods of Treating or Preventing a Condition in an Animal

The pharmaceutical compositions are useful in human medicine and veterinary medicine. The pharmaceutical compositions are particularly useful in veterinary medicine.

The invention further relates to a method of treating or preventing a condition in an animal. The method comprises administering to an animal in need thereof a pharmaceutical composition of the invention by injection. The injectable pharmaceutical composition can be administered, for example, by an intramuscular, intraperitoneal, or subcutaneous injection.

In one embodiment, the method involves treating a condition in an animal. In one embodiment, the method involves preventing a condition in an animal.

When the pharmaceutical compositions comprising (i) a dibasic amino acid ester or dibasic amino acid amide; (ii) a dicarboxylic acid; (iii) a pharmaceutically active compound or pharmaceutically acceptable salt thereof; and (iv) a pharmaceutically acceptable organic solvent are injected into an animal, the pharmaceutical compositions form a depot that provides sustained-release of the pharmaceutically active compound or pharmaceutically acceptable salt thereof. The components of the pharmaceutical composition, i.e., the dibasic amino acid ester or dibasic amino acid amide, the dicarboxylic acid, and the pharmaceutically acceptable organic solvent are biocompatible and non-toxic and, over time, are simply absorbed and/or metabolized by the body.

When the pharmaceutical compositions comprising micelles or liposomes suspended in an aqueous solvent, wherein the micelles or liposomes comprise (i) the dibasic amino acid ester or dibasic amino acid amide; (ii) the dicarboxylic acid; and (iii) the pharmaceutically active compound or pharmaceutically acceptable salt thereof are injected into an animal, the pharmaceutical compositions do not form a depot. Rather, the micelle or liposome structures are maintained, at least initially, after administration, and then overtime break down to release the pharmaceutically active compound, thus, providing sustained release of the pharmaceutically active compound.

Typically, the injectable pharmaceutical composition is injected in an amount of between about 0.2 mL and 15 mL, preferably between about 0.5 mL and 12 mL, more preferably between about 1 mL and 10 mL. The precise dose to be administered will depend on the seriousness of the condition, and the animal being treated and can be decided according to the judgment of a practitioner and/or each animal's circumstances. Smaller animals typically receive smaller injection volumes. For example, the injection volume for a cat is typically about 1 mL and the injection volume for a dog is typically between about 1 mL and 2 mL For large animals such as cows and horses, however, the injection volume can be as large as 10 mL and even larger. The amount of the pharmaceutical composition administered to an animal can be determined by standard clinical techniques. In addition, in vitro or in vivo assays can optionally be employed to help identify optimal dosage ranges.

In one embodiment, administering the pharmaceutical composition by injection provides an effective amount of the pharmaceutically active compound or pharmaceutically acceptable salt thereof in the blood or serum of the animal. The pharmaceutical compositions of the invention can provide an effective amount of the pharmaceutically active compound or pharmaceutically acceptable salt thereof in the blood or serum of the animal for a period of at least about 15 days, and even longer, depending on components of the pharmaceutical composition, i.e., the pharmaceutically active compound or pharmaceutically acceptable salt thereof, the dibasic amino acid ester or dibasic amino acid amide, the dicarboxylic acid, and the pharmaceutically acceptable organic solvent.

In one embodiment, the pharmaceutical composition provides an effective amount of the pharmaceutically active compound or pharmaceutically acceptable salt thereof in the blood or serum of the animal for at least about 2 days.

In one embodiment, the pharmaceutical composition provides an effective amount of the pharmaceutically active compound or pharmaceutically acceptable salt thereof in the blood or serum of the animal for at least about 3 days.

In one embodiment, the pharmaceutical composition provides an effective amount of the pharmaceutically active compound or pharmaceutically acceptable salt thereof in the blood or serum of the animal for at least about 4 days.

In one embodiment, the pharmaceutical composition provides an effective amount of the pharmaceutically active compound or pharmaceutically acceptable salt thereof in the blood or serum of the animal for at least about 6 days.

In one embodiment, the pharmaceutical composition provides an effective amount of the pharmaceutically active compound or pharmaceutically acceptable salt thereof in the blood or serum of the animal for at least about 8 days.

In one embodiment, the pharmaceutical composition provides an effective amount of the pharmaceutically active compound or pharmaceutically acceptable salt thereof in the blood or serum of the animal for at least about 10 days.

In one embodiment, the pharmaceutical composition provides an effective amount of the pharmaceutically active compound or pharmaceutically acceptable salt thereof in the blood or serum of the animal for at least about 12 days.

In one embodiment, the pharmaceutical composition provides an effective amount of the pharmaceutically active compound or pharmaceutically acceptable salt thereof in the blood or serum of the animal for at least about 15 days.

Advantageously, the pharmaceutical compositions, by providing sustained release of the pharmaceutically active compound or pharmaceutically acceptable salt thereof, have reduced toxicity, particularly in small animal such as cats and dogs. Accordingly, the pharmaceutical compositions of the invention have a better therapeutic profile that conventional immediate release formulations. The methods of the invention, which involve administering a pharmaceutically active compound or pharmaceutically acceptable salt thereof to an animal by injecting the animal with a pharmaceutical composition of the invention, permit pharmaceutically active compounds to be administered to animals that could, if administered in presently available dosage forms, result in toxicity and even death of the animal being treated. By providing sustained release of the pharmaceutically active compound or pharmaceutically acceptable salt thereof, the pharmaceutical compositions of the invention need to be administered less frequently and, therefore, are also easier to administer, more convenient, and more cost effective than conventional modes of administering pharmaceutically active compounds.

In another embodiment, the methods involves administering a pharmaceutical composition comprising a dibasic amino acid ester or dibasic amino acid amide, a dicarboxylic acid, and a pharmaceutically active compound (i.e., the pharmaceutically active compound entrapped in the extended salt structure) by a route other than injection. Other routes of administration include, but are not limited to, topical, oral, rectal, vaginal, and nasal. In one embodiment, the pharmaceutical composition is in the form of a solid, a crystal, a gummy mass, or a gel (i.e., it is not an injectable composition).

For example, encapsulating the pharmaceutical formulations in the form of a solid, a crystal, a gummy mass, or a gel in a capsule provides a dosage form that can be administered orally. Furthermore, solid pharmaceutical compositions of the invention can be combined with an excipient such as a binder, diluent, or lubricant and formulated into a tablet to provide a dosage form for oral administration. See, for example, Remington's Pharmaceutical Sciences, Alfonso Gennaro ed., 19th ed. 1995), incorporated herein by reference. Oral dosage forms can be designed to release the pharmaceutically active compound or pharmaceutically acceptable salt thereof in the stomach immediately or almost immediately or to provide sustained release of the pharmaceutically active compound or pharmaceutically acceptable salt thereof in the stomach. The rate of release of the pharmaceutically active compound or pharmaceutically acceptable salt thereof is varied by varying the lipophilicity and/or molecular weight of the components of the pharmaceutical composition.

Without wishing to be bound by theory, it is believed that when the solid pharmaceutical compositions of the invention are administered orally, the solid composition disintegrates when it reaches the stomach and forms micelles or liposomes comprising (i) the dibasic amino acid ester or dibasic amino acid amide; (ii) the dicarboxylic acid; and (iii) the pharmaceutically active compound or pharmaceutically acceptable salt thereof. The structure of the micelles or liposomes is believed to be similar to that depicted in FIG. 1. Without wishing to be bound by theory, it is believed that the micelle or liposome structures enhance the bioavailability of the pharmaceutically active compound or pharmaceutically acceptable salt thereof. The pharmaceutically active compound or pharmaceutically acceptable salt thereof can be a hydrophobic compound or a hydrophilic compound.

The pharmaceutical compositions comprising micelles or liposomes suspended in an aqueous solvent, wherein the micelles or liposomes comprise (i) the dibasic amino acid ester or dibasic amino acid amide; (ii) the dicarboxylic acid; and (iii) the pharmaceutically active compound or pharmaceutically acceptable salt thereof can also be administered orally.

Pharmaceutical compositions for oral administered are typically in the form of a capsule or tablet and typically contain between about 0.001 g and 2 g, preferably between about 0.01 g and 1.5 g, of the pharmaceutically active compound or pharmaceutically acceptable salt thereof.

Solid pharmaceutical compositions can also be administered by implanting a solid pharmaceutical composition containing between about 0.01 and 2 g, preferably between about 0.2 g and 1.5 g, of the pharmaceutically active compound or pharmaceutically acceptable salt thereof under the skin of the animal using methods well known to one of ordinary skill in the art.

The animal can be a human or a non-human.

In one embodiment, the animal is a human.

In one embodiment, the animal is a mammal.

In one embodiment, the animal is a canine, feline, equine, bovine, ovine, porcine, amphibian, reptile, or avian.

In one embodiment, the animal is a cat.

In one embodiment, the animal is a dog.

In one embodiment, the animal is a cow.

In one embodiment, the animal is a pig.

In one embodiment, the animal is a sheep.

In one embodiment, the animal is a horse.

### Kits

The invention encompasses kits that can simplify the administration of a pharmaceutically active compound or pharmaceutically acceptable salt thereof to an animal. A typical kit of the invention comprises a unit dosage form of a pharmaceutical composition of the invention. In one embodiment, the unit dosage form is a container, such as a vial, which can be sterile, containing a pharmaceutical composition of the invention. The kit can further comprise a label or printed instructions instructing the use of the pharmaceutically active compound to treat a condition. In another embodiment, the kit comprises a unit dosage form of a pharmaceutical composition of the invention and a syringe for administering the pharmaceutical composition.

The present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited, the entire disclosure of which are incorporated herein by reference.

What is claimed is:

1. A pharmaceutical compositions comprising:
(i) a dibasic amino acid ester or dibasic amino acid amide;
(ii) a dicarboxylic acid;
(iii) a pharmaceutically active compound or pharmaceutically acceptable salt thereof; and
(iv) a pharmaceutically acceptable organic solvent.

2. The pharmaceutical composition of claim 1, wherein the composition is injectable.

3. The pharmaceutical composition of claim 2, wherein the composition forms a precipitate when injected into an aqueous solution.

4. The pharmaceutical composition of claim 3, wherein the precipitate releases the pharmaceutically active compound over time.

5. The pharmaceutical composition of claim 2, wherein the composition forms a depot when administered to an animal by injection.

6. The pharmaceutical composition of claim 5, wherein the depot releases the pharmaceutically active compound over time.

7. The pharmaceutical composition of claim 1, wherein the dibasic amino acid ester is a dibasic amino acid vitamin ester.

8. The pharmaceutical composition of claim 1, wherein the dicarboxylic acid is an N-acylated amino acid, wherein the side chain of the amino acid contains an acidic functional group.

9. The pharmaceutical composition of claim 8, wherein the N-acylated amino acid is acylated with the residue of an acidic vitamin.

10. The pharmaceutical composition of claim 1, wherein the pharmaceutically acceptable organic solvent is selected from the group consisting of pyrrolidone, N-methyl-2-pyrrolidone, polyethylene glycol, propylene glycol (i.e., 1,3-propylene glycol, 1,2-propylene glycol, or a mixture thereof), glycerol formal, isosorbid dimethyl ether, ethanol, dimethyl sulfoxide, tetraglycol, tetrahydrofurfuryl alcohol, triacetin, propylene carbonate, dimethyl acetamide, dimethyl formamide, dimethyl sulfoxide, and combinations thereof.

11. The pharmaceutical composition of claim 1, wherein the amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid ranges from about 0.2 to 10 percent.

12. The pharmaceutical composition of claim 11, wherein the amount of the pharmaceutically active compound or pharmaceutically acceptable salt thereof as a weight percentage of the combined amount of the pharmaceutically active compound or pharmaceutically acceptable salt thereof, the dibasic amino acid ester or dibasic amino acid amide, and the dicarboxylic acid ranges from about 0.1 to 5.

13. The pharmaceutical composition of claim 1, wherein the molar ratio of the dibasic amino acid ester or dibasic amino acid amide to the dicarboxylic acid ranges from about 1.5:1 to 1:1.5.

14. The pharmaceutical composition of claim 1, wherein the combined amount of the dibasic amino acid ester or dibasic amino acid amide and the dicarboxylic acid is about 2 to 75 times more by weight than the amount of the pharmaceutically active compound.

15. The pharmaceutical composition of claim 14, wherein the combined amount of the dibasic amino acid ester or dibasic amino acid amide and the dicarboxylic acid is about 20 to 40 times more by weight than the amount of the pharmaceutically active compound.

16. The pharmaceutical composition of claim 1, wherein the combined amount of the pharmaceutically active compound or pharmaceutically acceptable salt thereof, the dibasic amino acid ester or dibasic amino acid amide, and the dicarboxylic acid ranges from about 1 to 50 percent by weight percent of the composition.

17. The pharmaceutical composition of claim 16, wherein the combined amount of the pharmaceutically active compound or pharmaceutically acceptable salt thereof, the dibasic amino acid ester or dibasic amino acid amide, and the dicarboxylic acid ranges from about 2 to 30 percent by weight percent of the composition.

18. The pharmaceutical composition of claim 4, wherein less than 20% of the pharmaceutically active compound is released in 6 hours when the aqueous solution is phosphate buffered saline.

19. The pharmaceutical composition of claim 18, wherein the less than 20% of the pharmaceutically active compound is released in 10 hours when the aqueous solution is phosphate buffered saline.

20. The pharmaceutical composition of claim 4, wherein the amount of pharmaceutically active compound released from the composition after 6 hours is less than the amount of pharmaceutically active compound that is released from a comparable composition that does not include the dibasic amino acid ester or dibasic amino acid amide or does not include the dicarboxylic acid.

21. The pharmaceutical composition of claim 4, wherein the amount of pharmaceutically active compound released from the composition after 10 hours is less than the amount of pharmaceutically active compound that is released from a comparable composition that does not include the dibasic amino acid ester or dibasic amino acid amide or does not include the dicarboxylic acid.

22. A method of treating or preventing a condition in an animal comprising administering to an animal in need thereof the pharmaceutical composition of claim 1.

23. The method of claim 22, wherein the administering is by injection.

24. The method of claim 23, wherein administering the pharmaceutical composition forms a depot in the animal that releases the pharmaceutically active compound or pharmaceutically acceptable salt thereof over time.

25. The method of claim 23, wherein the pharmaceutical composition provides an effective amount of the pharmaceutically active compound or pharmaceutically acceptable salt thereof in the blood or plasma of the animal for at least about 2 days.

26. A pharmaceutical composition comprising micelles or liposomes suspended in an aqueous solvent, wherein the micelles or liposomes comprise
(i) a dibasic amino acid ester or dibasic amino acid amide;
(ii) a dicarboxylic acid; and
(iii) a pharmaceutically active compound or pharmaceutically acceptable salt thereof.

27. The pharmaceutical composition of claim 26, wherein the dibasic amino acid ester is a dibasic amino acid vitamin ester.

28. The pharmaceutical composition of claim 26, wherein the dicarboxylic acid is an N-acylated amino acid, wherein the side chain of the amino acid contains an acidic functional group.

29. The pharmaceutical composition of claim 28, wherein the N-acylated amino acid is acylated with the residue of an acidic vitamin.

30. A method of treating or preventing a condition in an animal comprising administering to an animal in need thereof the pharmaceutical composition of claim 26 by injection.

31. A method of treating or preventing a condition in an animal comprising orally administering to an animal in need thereof the pharmaceutical composition of claim 26.

32. A solid pharmaceutical composition comprising
(i) a dibasic amino acid ester or dibasic amino acid amide;
(ii) a dicarboxylic acid; and
(iii) a pharmaceutically active compound or pharmaceutically acceptable salt thereof wherein the composition forms micelles or liposomes when reconstituted with water.

33. A method of treating or preventing a condition in an animal comprising orally administering to an animal in need thereof the pharmaceutical composition of claim 32.
